(19) United States
(12) Patent Application Publication
(10) Pub. No.: US 2008/0176266 A1
(43) Pub. Date: Jul. 24, 2008

(54) Biomarkers Of Metabolic Responses To Hepatotoxicants And Carcinogens

(76) Inventors: Alvin Berger, Raleigh, NC (US); Imran A. Shah, Chapel Hill, NC (US)

Correspondence Address: HUTCHISON LAW GROUP PLLC, PO BOX 31686, RALEIGH, NC, 27612, US

(21) Appl. No.: 11/840,464

(22) Filed: Aug. 17, 2007

Related U.S. Application Data
(60) Provisional application No. 60/838,561, filed on Aug. 17, 2006.

(51) Int. Cl. C12Q 1/02

(52) U.S. Cl. 435/29

(57) ABSTRACT

Methods for the measurement and prediction of response to hepatotoxicants and carcinogens through the detection of metabolites in a mammal are provided. The metabolites can be used as biomarkers, including efficacy biomarkers, surrogate biomarkers, and toxicity biomarkers. The methods find use for early prediction of toxicity, target identification/validation, and monitoring of drug efficacy.

### CROSS-REFERENCE TO RELATED APPLICATIONS

This application claims the benefit of U.S. Provisional Application No. 60/838,561, filed Aug. 17, 2006, the entirety of which is hereby incorporated by reference.

### STATEMENT REGARDING FEDERALLY SPONSORED RESEARCH

This invention was made with United States Government support under SBIR Phase I Contract #: 291200445524C (SBIR Phase I Contract Solicitation PHS-2004-1-100) awarded by the National Institute of Environmental Health Sciences (NIEHS). The United States Government has certain rights in the invention.

### FIELD

The invention relates generally to methods of measuring metabolic responses to hepatotoxicants and carcinogens.

### BACKGROUND

Hepatotoxicants and carcinogens have been studied for metabolic effect prior to the omic era, and more recently using microarray transcriptomic technology. While the latter approach has greatly expanded knowledge of such compounds, transcriptomic approaches do not actually measure the metabolites (small molecules) and pathways perturbed.

For example, clofibrate is a fibrate type of hypolipidemic drug, and also a hepatotoxicant and carcinogen. It acts on peroxisome proliferator activated receptor alpha (PPARα) receptors. Peroxisome proliferator activated receptors (PPARs) are nuclear hormone receptors that are activated by micromolar concentrations of lipids, fibrates and thiazolidinediones. This subfamily can be divided into three isotypes, designated PPARα, β, and γ, each with tissue-specific expression. PPARα receptors are particularly abundant in rodents, but also present in humans. In humans, PPARγ predominates over PPARα, and hepatocyte nuclear factor (HNF) has some similar functions as PPARα in rodents, but both PPAR types are present in rodents and humans. Clofibrate (ethyl-p-chloro-phenoxyisobutyrate; CAS 637-07-0)) is a fibrate type of hypolipidemic (cholesterol lowering) drug, which is also a hepatotoxicant and carcinogen at high levels. It acts predominately on PPARα receptors.

Clofibrates work by activating PPARs, which in turn form heterodimers with retinoid X receptor (RXR), and interact with the peroxisome proliferator response element (PPREs) in gene promoters. PPREs are direct repeats (DR) of a hexanucleotide sequence AGGTCA separated by one nucleotide and are therefore referred to as a DR-1 response element. PPARα and PPARγ play critical roles in the catabolism and storage of fatty acids, whereas the function of PPARδ is less certain. PPARα is the predominant PPAR subtype expressed in liver.

The overall effects of clofibrate are to decrease fat synthesis and increase fat degradation; and to decrease glycolysis and increase gluconeogenesis. In essence, clofibrate mimics the fasted metabolic state. Other effects of clofibrate observed in some studies are: increased oxidative stress; increased cell replication; and increased spontaneous preneoplastic lesions. Short term treatment of clofibrate may not induce transcriptional events as efficiently or at all, as no DNA adducts have been observed. Gonzalez et al. (1998) J Natl Cancer Inst 90: 1702-1709. PPARα regulate genes involved in fatty acid transport, synthesis and oxidation, glucose and lipid metabolism, ketogenesis and Δ5, Δ6, and Δ9-desaturation of fatty acids. Specific genes altered by clofibrate, with possible PPREs are described in Berger et al. (2002) Lipids Health Dis 1: 2 and Hamadeh et al. (2002) Toxicol Sci 67: 219-231.

Clofibrate has been studied at high doses for various durations, for its hepatotoxic and carcinogenic effects with microarrays, thus providing a putative map of how clofibrate may affect metabolism. In one study, rats exposed to clofibrate were monitored over time by a combination of histopathology and a transcriptomic approach. After 24 h, there were no microscopic changes to liver after a single exposure of clofibrate or other toxicants. In contrast, after 2 weeks, clofibrate induced hypertrophy. Although a similar set of genes was modified under both conditions, pattern recognition could distinguish the different drug treatments.

These studies demonstrate the predictive biomarker potential of hepatic transcriptomics with respect to liver histopathology changes in response to exposure to hepatotoxicants and carcinogens. Nonetheless, such approaches fail to actually measure the metabolites and pathways perturbed. Thus, there is a need for readily accessible biomarkers of exposure to hepatotoxicants and carcinogens (i.e., biomarkers present in serum, blood, or saliva).

### SUMMARY

Methods are provided for the measurement and prediction of response to hepatotoxicants and carcinogens through the detection of metabolites in a mammal. Such metabolites are useful as biomarkers, including efficacy biomarkers, surrogate biomarkers, and toxicity biomarkers.

In one embodiment, the metabolites are obtained from tissue. In one embodiment, the metabolites are obtained from a bodily fluid. In one embodiment, the metabolites are obtained from liver. In one embodiment, the metabolites are obtained from blood. In one embodiment, the metabolites are obtained from serum.

### BRIEF DESCRIPTION OF THE FIGURES

FIG. 1. The distribution of the coefficient of variation (CV) for the relative intensity of each LC-MS component for each machine replicate for the Day 1 liver samples is shown.

FIG. 2. Hierarchical agglomerative clustering of the metabolomic profiles from the liver samples of d 1 subjects showing clustering of technical replicates together as a measure of data quality.

FIG. 3. Four visualizations are shown of the PCA for the serum and liver data from day 1 and day 14 subjects.

FIG. 4. Four hierarchical agglomerative clusterings of serum and liver metabolomic profiles from (a) day 1 rat serum, (b) day 14 rat serum; (c) day 1 rat liver; and (d) day 14 rat liver.

FIG. 5. Network analysis results from the putatively identified metabolites that are correlated with liver hypertrophy. The visualization shows the metabolites as circles and biochemical interactions as lines connecting the circles in a network. A subset of these metabolites is set forth in the Detailed Description, Table 2.

### DETAILED DESCRIPTION

Methods are provided for determining exposure of a cell or cells to a chemical compound. In one embodiment, the method comprises the steps of measuring a biomarker panel of one or more metabolites in a sample taken from cell or cells; combining the measurements for the metabolites using a mathematical function including the measurements; and obtaining and analyzing an output from the function, wherein the output of the function is indicative of exposure to the chemical compound. In one embodiment, the chemical compound is a hepatotoxicant. In one embodiment, the chemical compound is a carcinogen through the detection of metabolites. In one embodiment, the cell or cells are in vivo. In one embodiment, the cell or cells are in vitro. In one embodiment, the cell or cells are mammalian.

Methods are also provided for constructing the function from a dataset comprising metabolite measurements taken from a plurality of samples. The samples may be from groups displaying differing phenotypes, or from groups subject to differing doses or times of exposure to a chemical compound. In one embodiment, the function is constructed by a statistical method followed by a step of performance evaluation. In one embodiment, the function is obtained by multivariate analysis of the dataset. Techniques of multivariate analysis are known and are discussed in Dillon & Goldstein, Multivariate Analysis: Methods and Applications, John Wiley & sons, New York (1984) and Duda, Hart, & Stork, Pattern Classification, 2d ed., John Wiley & Sons, New York (2000), each of which is incorporated herein by reference in its entirety. Performance of the function can be evaluated by various statistical methods. The output of such a method is metabolites that can serve as biomarkers, including efficacy biomarkers, surrogate biomarkers, and toxicity biomarkers.

The metabolites are detected using analytical chemistry techniques, including mass spectrometry. In one embodiment, the metabolites are detected using gas chromatograph-mass spectrometry (GC-MS). In one embodiment, the metabolites are detected using liquid chromatograph-mass spectrometry (LC-MS). GC-MS techniques are known in the art, including without limitation Quadrupole GC-MS, Ion-trap GC-MS, Time-of Flight GC-MS, Sector GC-MS, etc. LC-MS techniques are known in the art, including without limitation Quadrupole Triple LC-MS, Quadrupole LC-MS, 3D-Ion-trap LC-MS, Linear Iontrap LC-MS, Time-offlight LC-MS, Quadrupole-Time-offlight LC-MS Hybrid LC-MS, Sector LC-MS, FT-ICR LC-MS, etc. MALDI-TOF MS techniques are known in the art, including without limitation Linear only MALDI-TOF MS, Linear and/or Reflectron MALDI-TOF MS, TOF-TOF MALDI-TOF MS, etc. Such techniques are reviewed in Burlingame et al. (2000) Mass Spectrometry In Biology & Medicine Totowa, N.J., Humana Press; Niessen (2001) Current Practice Gas Chromatography-Mass Spectrometry, Marcel Dekker Inc., New York, N.Y.; W. Niessen (1998) Liquid Chromatography-Mass Spectrometry 2d Ed., Marcel Dekker Inc., New York, N.Y.; and Imma Ferrer et al., American Chemical Society (2003) Liquid Chromatography/Mass Spectrometry MS/MS and Time of Flight MS: Analysis of Emerging Contaminants, each of which are incorporated by reference herein in their entirety. As is known to those of skill in the art, the output of mass spectrometry is a peak characteristic of a given chemical compound or compounds (including metabolites). Until it is assigned an identity, each mass spectrometry is termed a component.

In one embodiment, the hepatotoxicant is clofibrate. Clofibrate or vehicle is administered orally (0, 50, 250 mg/kg/d) to groups of 6 rats and serum and livers are collected 6 and 24 h after either a single or 14 daily doses. Global biochemical profiles are determined by LC-MS and GC-MS and components highly perturbed by clofibrate exposure are identified by the methods described above. One or more of the biomarkers identified in the present methods may be utilized as biomarkers of clofibrate exposure. These are provided in Table 1(a) (Serum) and Table 1(b) (Liver).

TABLE 1(a)
Serum Early Components

| Components | Putative Identities |
|---|---|
| n_201.02_541 | Bergaptol |
| | Xanthotoxol |
| | 2,2′,3-Trihydroxybiphenyl |
| | 4-Carboxy-2-hydroxy-cis,cis- |
| | muconate |
| | (E)-4-Oxobut-1-ene-1,2,4- |
| | tricarboxylate |
| | 4-Carboxy-2-hydroxyhexa-2,4- |
| | dienedioate |
| | 4-Carboxy-2-oxo-3-hexenedioate |
| | Benzoyl phosphate |
| n_203.07_379 | 5-L-glutamyl-glycine |
| | 1,2-dipropanoyl-‘sn’-glycerol |
| | L-Tryptophan |
| | D-Tryptophan |
| | N-Acyl-D-mannosaminolactone |
| | Diethyl (2R,3R)-2-methyl-3- |
| | hydroxysuccinate |
| | Diethyl (2S,3R)-2-methyl-3- |
| | hydroxysuccinate |
| | Oxaloglutarate |
| | Dimethylenetriurea |
| | Spirodilactone |
| | Triethylenemelamine |
| | Droserone |
| | Vasicinol |
| | Idazoxan |

TABLE 1(b)
Liver Early Components

| Components | Putative Identities |
|---|---|
| n_114.05_94 | Acetamidopropanal |
| | L-proline |
| | L-Proline |
| | D-Proline |
| n_267.08_90 | Homocystine |
| | Acetylcarnosine |
| | Inosine |
| | Inosine |
| | Homocystine |
| | Lysergic acid |
| | Portulacaxanthin III |
| | 8-Azaadenosine |
| | 2(alpha-D-Mannosyl)-D-glycerate |

Table 1. The above table shows components significantly perturbed (p<0.01) at 6 h that return to baseline levels by 24 h in liver and in serum following day 1. Table 1(a) shows day 1 serum components; Table 1(b) shows the components found in liver on day 1.

In one embodiment, the metabolites correlate with a particular liver pathology. Thus, methods are provided for the prediction of liver pathology through the detection of metabolites. Components that correlate with liver hypertrophy are useful as surrogate endpoints of liver hypertrophy in the methods described herein. These are set forth in Table 2.

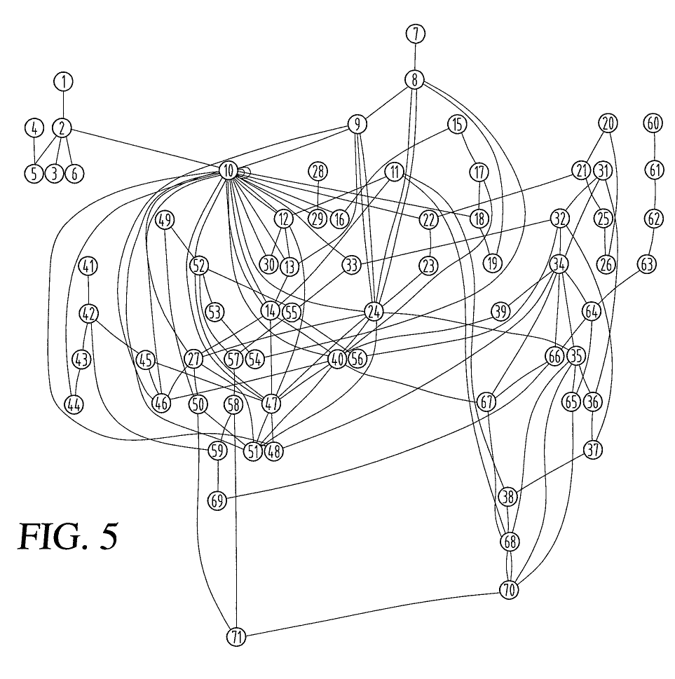

TABLE 2
Seven LC-MS components and their putative identities, associated by
number with the metabolic network of FIG. 5, above.

| Node | Component | Identification |
|---|---|---|
| 1 | p_316.18_464 | (S)-Nororientaline |
| 3 | p_316.18_464 | (S)-Norreticuline |
| 4 | p_316.18_464 | (R)-Norreticuline |
| 6 | p_316.18_464 | Nororientaline |
| 7 | n_462.15_87 | Lysosomal-enzyme N-acetyl-D-glucosaminyl- |
| | | phospho-D-mannose |
| 11 | n_462.15_87 | N6-(1,2-Dicarboxyethyl)-AMP |
| 15 | n_351.04_170 | Arbutin 6-phosphate |
| 20 | n_351.04_170 | 4-(4-Deoxy-alpha-D-gluc-4-enuronosyl)-D- |
| | | galacturonate |
| 25 | n_351.04_170 | 4-(4-Deoxy-beta-D-gluc-4-enuronosyl)-D- |
| | | galacturonate |
| 27 | n_322.07_90 | CMP |
| 28 | n_321.06_80 | N-((R)-Pantothenoyl)-L-cysteine |
| 30 | n_321.06_80 | dTMP |
| 31 | n_288.08_173 | N-Succinyl-2-L-amino-6-oxoheptanedioate |
| 41 | n_252.05_80 | Neopterin |
| 49 | n_252.05_80 | Diacylglyceryl-2-aminoethylphosphonate |
| 60 | n_252.05_80 | 5-(3-Carboxy-3-oxopropenyl)-4,6- |
| | | dihydroxypicolinate |

Thus, in one embodiment, methods are provided for the prediction of liver hypertrophy through the detection of metabolites. Because liver hypertrophy is a known marker of toxicity, these metabolites are useful as biomarkers of toxicity.

In one embodiment, the metabolites correlate with drug efficacy. Clofibrate, for example, acts on PPARα and has hypolipidemic effects. Clofibrate acts to decrease fat synthesis, increase fat degradation, decrease glycolysis and increase gluconeogenesis. Metabolites changing via PPARα cascades are potential efficacy markers. Using the present methods, metabolites were identified that changed with clofibrate exposure. These metabolites are set forth in Table 3.

TABLE 3
Hepatic efficacy biomarkers identified by GC-MS in rats exposed to 250 mg/kg/d (fold change)

| | | NIST | | | | |
|---|---|---|---|---|---|---|
| | | SIMILARITY | DAY 1 | | DAY 14 | |
| CLASS | Compound Name | SCORE | 6 H | 24 H | 6 H | 24 H |
| Amino acid | Glycine | 846 | 2.69** | 3.42* | | |
| | l-Alanine | 907 | | 4.74* | | |
| | L-Aspartic acid | 831 | | 27.88*** | | |
| Fatty | Palmitate | 898 | 0.95*** | | 0.72** | |
| Acid | Stearate | 856 | 1.07* | | | |
| | Linoleic acid | 914 | 0.97* | | | |
| | Arachidonic acid | 921^{a} | | | 0.22*** | |
| | Docosahexaenoic acid | 826 | | | | 0.58* |
| Carbohydrate | Glucose (aq) | 898 | 0.29* | | | 0.39** |
| Metabolism | Lactic acid | 930 | 1.56** | 4.41* | | |
| | Succinic acid (Butanedioic | 857 | | 17.05** | | |
| | acid) | | | | | |
| | Malate | 747 | | | 0.1** | 0.33** |
| MAG | Glycerol 1(3)-phosphate | 879 | | 2.56* | | |
| metabolism | 1-Mono | 849 | 0.91** | | 0.04** | |
| | palmitoylglycerol | | | | | |
| | 1-Mono | 838 | | 17.14** | 0.22** | |
| | stearoylglycerol | | | | | |
| | 2-Mono | 807 | | | 0.41* | |
| | stearoylglycerol | | | | | |
| | 1-Mono | Manual | 0.98* | | | |
| | oleoylglycerol | | | | | |
| | 2-Mono | 799 | 1.12*** | | | |
| | oleoylglycerol | | | | | |
| | 1-Mono | Manual | 1.11*** | | | |
| | linoleoylglycerol | | | | | |
| Sterol | Cholesterol | 864^{a} | 1.34*** | | 1.87** | 0.69** |
| | β-Sitosterol | 746^{a} | | | 0.37** | 0.46* |

| | | | |
|---|---|---|---|
| Clofibrate | Propanoic acid, 2-(4- | 869 | (not present in control) |

| | |
|---|---|
| metabolite | chlorophenoxy)-2-methyl- |

In one embodiment, methods are provided for the characterization of clofibrate efficacy through the detection of metabolites. Because clofibrate is itself a PPAR activator, these metabolites are useful as biomarkers of PPAR activators.

Because the biomarkers provided in Table 2 correlate with toxicity and the biomarkers of Table 3 correlate with efficacy, they are useful in methods for separating on-target from off-target drug effects through the detection of metabolites.

The following examples are offered by way of illustration and not by way of limitation.

### EXAMPLES

### Example 1

### Design of Clofibrate Studies

A. Study 1:

Rats (6 per group) were dosed by gavage with vehicle, 50 or 250 mg/kg clofibrate per day for 1 (single dose) or 14 days (repeated dose). These groups are referred to as “day 1” and ‘day 14 (d 1 and d14).” Liver and serum were collected at 6 and 24 h post-dosing in the day 1 and day 14.

In detail, a single dose of clofibrate or vehicle was administered by gavage (0, 50 and 250 mg/kg) to groups of 18 male Sprague Dawley rats at 11 wks age. Six rats per dose group were euthanized at 6 and 24 h post-dose. Serum (for metabolomics and alanine aminotransferase (ALT), and aspartate aminotransferase (AST)), liver lobes (for histology), and frozen liver and urine (for metabolomics) were collected. Six rats per dose group were placed in metabolism cages for urine collection at −24-0 h, 0-6 h, 6-24 h and 24-48 h. These rats were removed from metabolism cages at 48 h post-dose, for blood and liver collection. The detailed study design is set forth in Chart 1.

CHART 1
Detailed study design

| Study | | | Doses | | |
|---|---|---|---|---|---|
| designation in | | # Rats/ | clofibrate | | |
| protocol | Organ | group | (0, 50, 250) | Details of Time pts | Total |

Single exposure study (1 injection in 1 d)

| | | | | | |
|---|---|---|---|---|---|
| study 1 (d 1) | liver | 6 | 3 | 6 h, 24 h after dosing | 36 |
| study 2 (d 1) | liver | 6 | 3 | 48 h | 18 |
| study 1 (d 1) | plasma | 6 | 3 | 6, 24 h | 36 |
| study 2 (d 1) | plasma | 6 | 3 | 48 h | 18 |
| study 2 (d 1) | urine | 6 | 3 | −24-0 (baseline), 0-6, 6-24, 24-48 | 72 |

Repeated exposure study (13 injections in 14 d, measurements over last 2 d)

| | | | | | |
|---|---|---|---|---|---|
| study 1 (d 14) | liver | 6 | 3 | 14 d + 6 h, 14 d + 24 h, | 36 |
| study 2 (d 14) | liver | 6 | 3 | 14 d + 48 h | 18 |
| study 1 (d 14) | plasma | 6 | 3 | 14 d + 6 h, 14 d + 24 h, | 36 |
| study 2 (d 14) | plasma | 6 | 3 | 14 d + 48 h | 18 |
| study 2 (d 14) | urine | 6 | 3 | 14 d + 6 h (0-6), 14 d + 24 h (6-24), | 54 |
| | | | | 14 d + 48 h (24-48) | |

B. Study 2:

Another group of rats received 14 repeated daily gavaged doses (0, 50 and 250 mg/kg) of vehicle or clofibrate. These rats were transferred to metabolism cages following administration of either 1 or 13 doses of clofibrate at 50 mg/kg, 250 mg/kg, or by vehicle. Urine was collected at various time points (Chart 1). Urine was not collected at time point −24-0 (baseline) at day 14 (see study 2 (d 14)). The protocol was nearly identical to the single dose experiment.

### Example 2

### Sample Preparation And Evaluation

Samples were extracted with 20% acetonitrile, then were evaporated and re-constituted in distilled water. For the liver, the left lobe was selected for metabolomic analysis.

Liver enzymes: ALT and AST were not elevated in any groups.

Histology: After a single dose, there was a dose related increase and severity of hepatocellular mitotic figures as dose increased from 50 to 250-mg/kg. After 14 doses at 250 mg/kg/day, hepatocellular cytologic alterations (indicating loss of glycogen and eosinophilic granular cytoplasm) were noted at all time points.

### Example 3

### LC-MS Metabolomics

LC-MS was performed in positive and negative electrospray ionization modes on Bruker time of flight (TOF) instruments, using Icoria™ proprietary HPLC methods and picking and alignment programs. Samples were randomly placed in wells on 96-well plates, keeping d 1 and d 14 samples on separate plates. Between 54 and 72 samples plus quality control samples were run on each plate. Pre- and post-flight instrument checks were carried out. Thereafter, data integrity checks were performed to detect any errors related to our Laboratory Information Management System (LIMS) system, labeling of samples, and missing or extraneous information.

### Example 4

### Computational And Statistical Procedures

The quality of the metabolomic data was visually assessed through the distribution of the coefficient of variation for each aligned LC-MS component across technical replicates and the reproducibility of the metabolomic profiles was evaluated by clustering the technical (machine) replicates. A component represents a single molecule or a group of molecules with very similar structural similarity (e.g., an isomer) that bin together on the m/z-retention time grid during alignment of peaks. A technical replicate refers to an aliquot of the same sample plated on different wells of a plate, in random fashion (as opposed to an independent extraction of the same sample).

Trends between dose and time points, for serum and liver, were assessed using two techniques. First, principal components analysis (PCA) was used to visually assess biological variability (as proposed originally). Furthermore, an unbiased quantitative assessment of the separation between the subjects in each dose-time group was conducted using an unsupervised learning approach based on hierarchical agglomerative clustering of the metabolomic profiles for the subjects.

T-tests were conducted to identify the significantly perturbed components in the liver and the serum of subjects at each time post-dose by comparison against the control subjects (vehicle control group). F-tests were conducted to identify those metabolomic components that were significantly perturbed in response to dose, time, and dose-time interaction.

Metabolites associated with LC-MS components were putatively identified using our proprietary database of mammalian metabolites as well as external sources of metabolic information.

### Example 5

### Identification

A series of standards were run and their retention time, m/z, and intensity were stored in a database of components. Components identified above were then compared to this known component database. Additionally, internal and external compound databases (Brenda, Kegg, ChemFinder) were queried for similarities in exact mass, and then to eliminate xenobiotic molecules or molecules that were not reasonable from a polarity perspective.

### Example 6

### LC-MS Metabolomic Data Generation

LC-MS peaks from each sample were aligned by mass to charge (m/z) ratio and retention time (RT) across all samples for each matrix, and quantified using Icoria's proprietary software. LC-MS components for each replicate of each sample were represented mathematically as a vector. Each component in each sample has three associated measurements: raw intensity in each sample; chromatographic retention time for peaks; and the mass divided by the charge (M/z). The metabolomic profile of each sample (denoted as x) is defined by the set of all components of known intensity, retention time and mass divided by charge. Before analyzing the data for we conducted the following preprocessing steps:

Step 1. Normalization to internal standard: Each metabolomic profile, x, was normalized using the intensity of a standard compound (called the ‘internal standard’, which was added to each sample), transforming x into a relative intensity profile. This step is necessary to address the systematic variation of raw intensity measurements between samples due to instrument signal fluctuation. For this purpose, the internal standard need not be chemically and structurally related to the metabolites of interest. Three internal standard are added to each matrix and the internal standard giving the most consistent responses (best separation in m/z axis, less matrix suppression, best peak shapes, etc.) is selected. These were d3 methionine for liver and serum, and d5 tryptophan for urine.

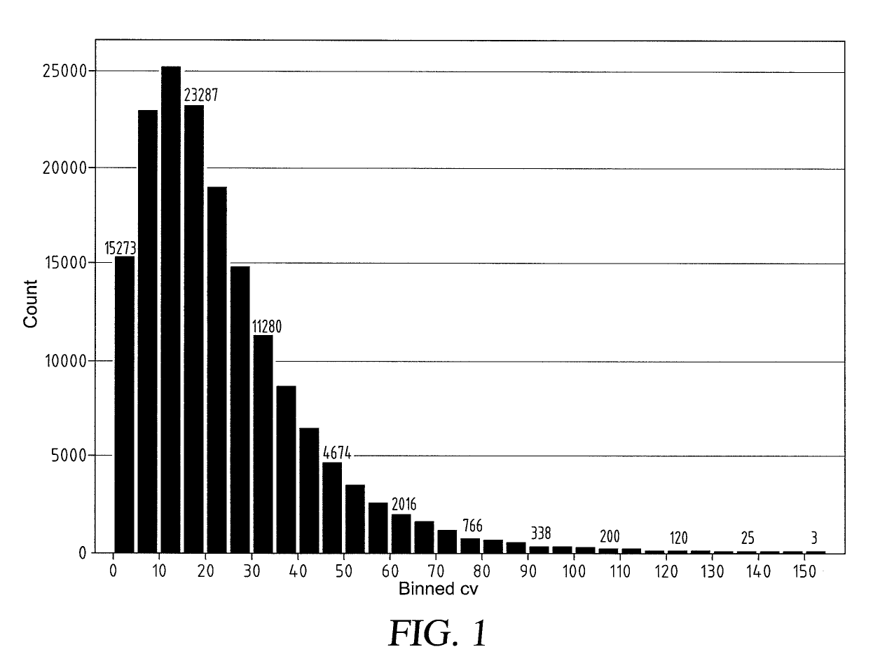

Step 2. Technical variability and average metabolomic profile: The technical (machine) replicate variation in components was measured using the coefficient of variation, CV, of the relative intensities (where

$${CV}_{jk} = \frac{{σ}_{jk}}{{μ}_{jk}} ,$$

where σⱼₖ=standard deviation of component k in sample j, μⱼₖ=mean of component k in sample j). The mean value of the relative intensity, μⱼₖ, for each component is used to build the average metabolomic profile for each subject. FIG. 1 shows the distribution of the CV across all samples (for day 1 liver samples). Although some components had a very high CV in some samples, median CV was between 10-20% with the majority of components having much lower values.

Step 3. Missing value correction: Components that were not observed across the three machine replicates were treated stringently, using deletion. If the component was observed across all replicates, μⱼₖ was calculated using three relative intensity values. When the component was observed in only two subjects, μⱼₖ was calculated between two observed values. When the component was only observed in one replicate, μⱼₖ was set to the limit of detection (a low intensity value).

Step 4. Distribution of relative intensity and log transformation: Though the literature on the intensity distribution of metabolomic data is limited, in our studies we have found this close to the lognormal probability density function. There are two main reasons to consider this transformation. Biologically, this transformation enables consideration of low concentration metabolites that capture subtle but important effects. Statistically, this transformation is important for measuring the similarity between the biochemical profiles of samples (through a distance metric). Hence, we analyze the intensity distribution in the biochemical profiles of the samples for skewness visually and transform logarithmically (base e and 10) if it is lognormal.

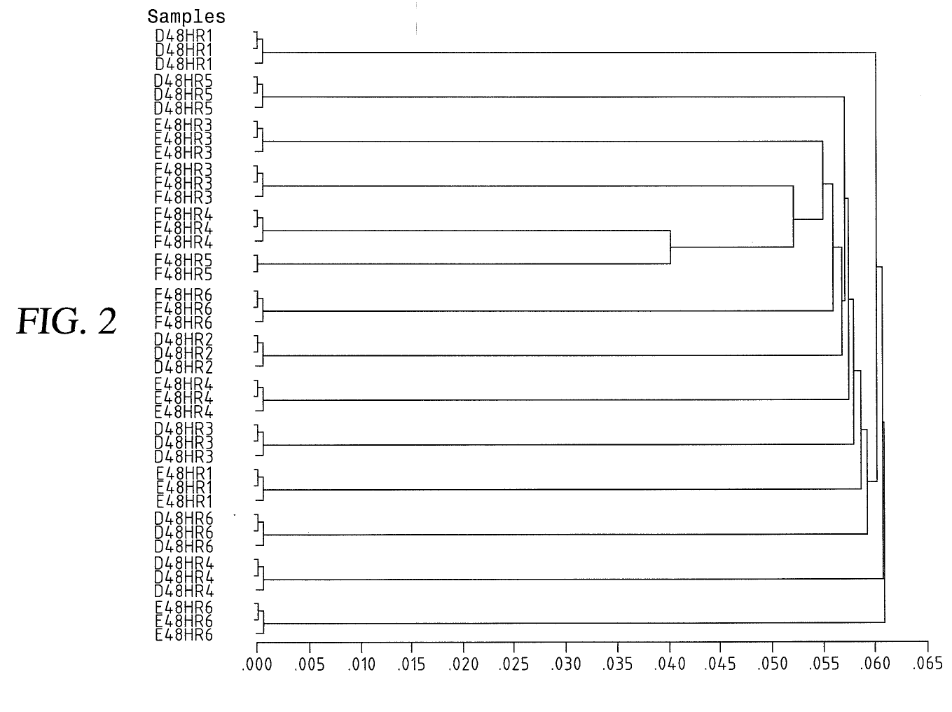

Step 5. Data quality: clustering of technical replicates: In addition to the quality control procedures described in Section IV. B. we assessed quality of replication by comparing metabolomic profiles for each subject from each tissue by using hierarchical agglomerative clustering using Pearson correlation as the distance metric. Technical replicates were found to group together consistently. For example, FIG. 2 shows the grouping of a randomly selected subset of the technical replicates for day 1 liver samples. The total number of components observed on d 1 and d 14 across liver and serum are given in Chart 2 below. Our current metabolomic profiling extraction and mass spectral conditions favor the presence and detection of polar metabolites.

Chart 2. Shown are the total number of metabolomic components observed by LC-MS in liver and serum on d 1 and d 14. The number of components shown for each tissue would not necessarily be the same. Each value includes an average of 175 components present in the blank that can be subtracted off. There appears to be more components detected in d 1 as compared to d 14 for each matrix; and more components in liver relative to serum, at each time point.

| | |
|---|---|
| Day 1 | Day 14 |

| Liver | Serum | Liver | Serum |
|---|---|---|---|
| 6764 | 5345 | 6599 | 4895 |

### Example 7

### Dose And Temporal Data Trends

An unbiased grouping of subjects using liver and serum data for days 1 and 14 was analyzed as described in the following paragraphs.

### Dose And Time Effects Studied With PCA

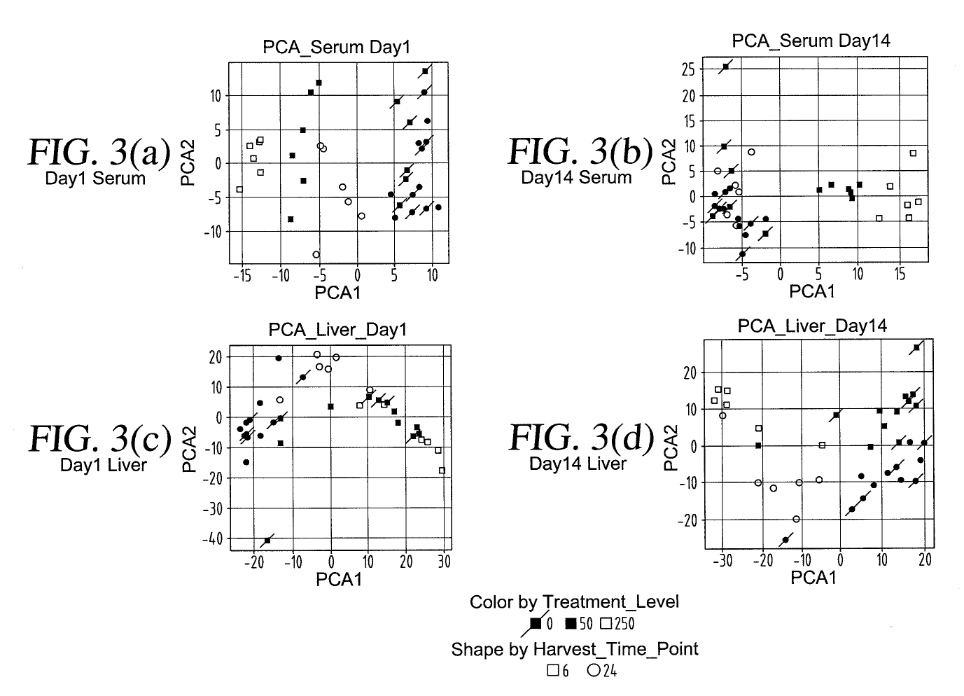

Dose and time effects were first studied with PCA to visually assess data groupings. Serum and liver days 1 and 14 are shown in FIG. 3. FIG. 3(a) shows separation of groups by both dose and time. FIG. 3(b) shows that in day 14 serum, the 50- and 250 mg/kg dose groups are separate from the rest of groups. FIGS. 3(c) and 3(d) show a similar trend in the liver data. In day 1 serum, there was separation of groups by both dose and time after drug administration (FIG. 3(a)). Control treated rats (red rectangles and circles) are not particularly well separated spatially, particularly in PCA 1. In day 14 serum, 50 and 250 mg doses are separated from one another and all other groups after 6 h, but all other groups are not well separated (FIG. 3(b)). The fact that 50 and 250 mg doses are not well separated from controls after 24 h (compare red, blue, and yellow circles), may be a first indication of some adaptive response to return to homeostasis within 24 h post gavage, when the drug was administered chronically for 14 days. In distinct contrast to serum, in day 1 liver, there was not a clear separation of groups on the basis of dose and post-gavage time (FIG. 3(c)). In day 14 liver, there was some tendency for the high dose 250 mg dose group to separate from other groups (yellow squares and circles), but other trends are less clear (FIG. 3(d)).

### Effects of Dose And Time Studied With Hierarchical Clustering

Hierarchical agglomerative clustering was used with Ward's minimum variance method, with correlation as the distance metric to discover natural data groupings. Generally, sub-clusters separated by dose and time. The two principal clusters for each day and time are described below.

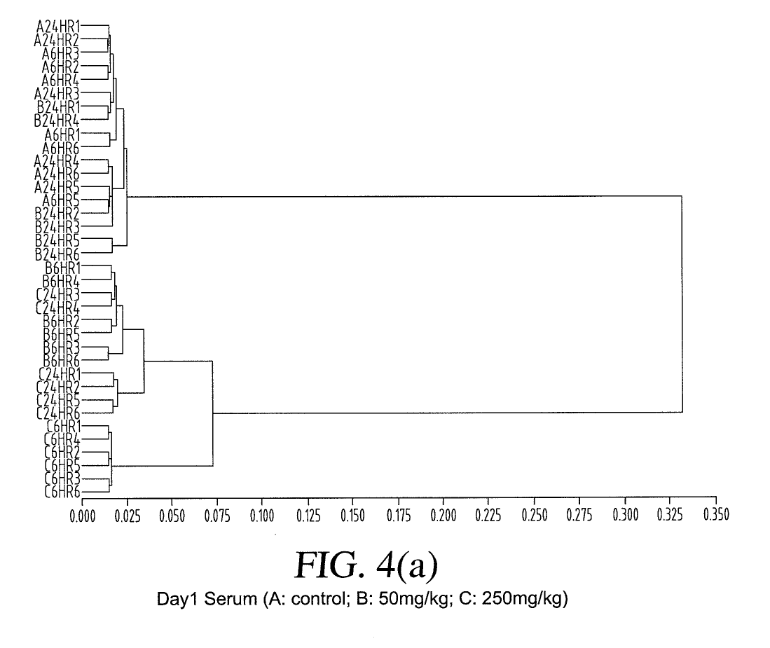

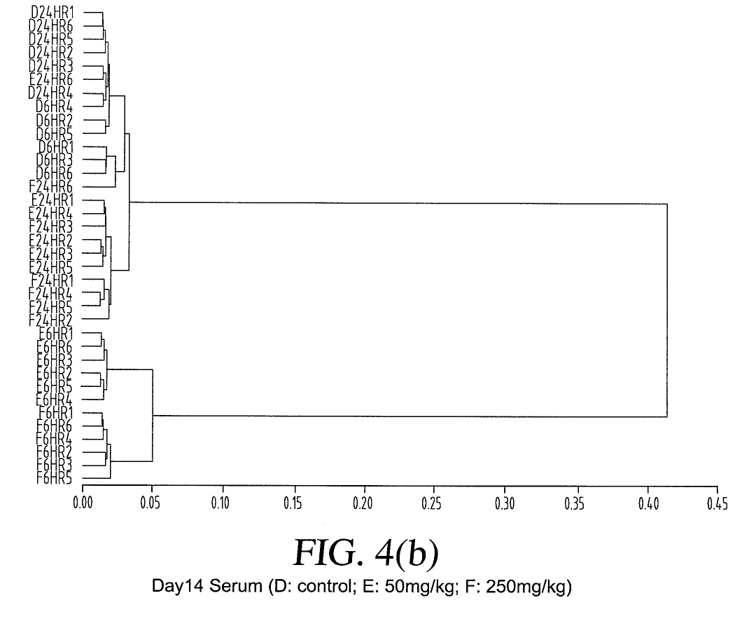

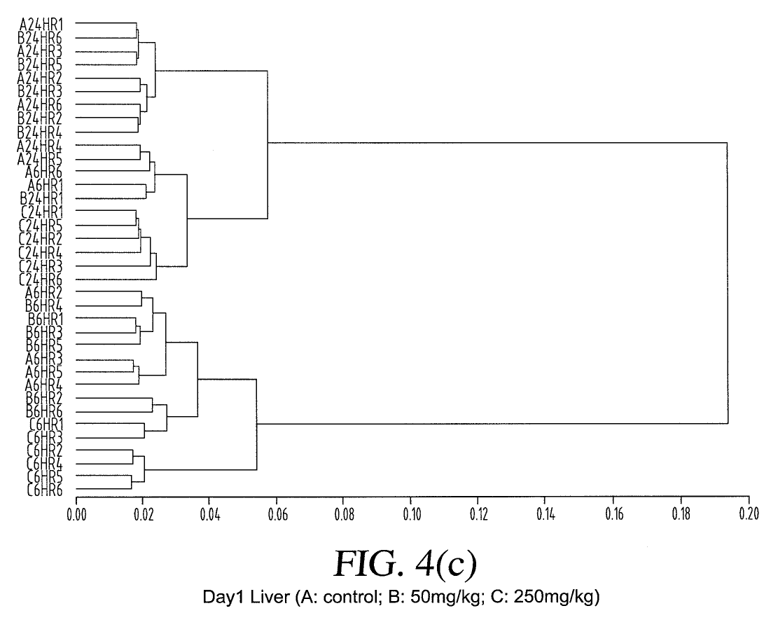

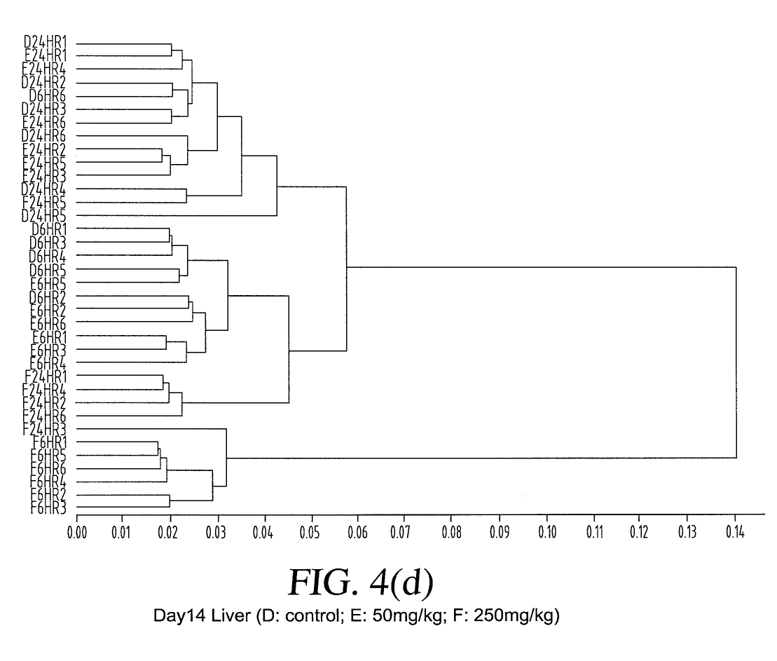

In day 1 serum samples, there were two main clusters: cluster 1 was comprised of control (A6H and A24H) and 50 mg dose at 24 h (B24H); cluster 2 was comprised of 50 mg dose at 6 H (B6H) and 250 mg dose at 6 and 24 h (B24H, C6H, C24H)(FIG. 4(a)). This would indicate some tendency for return to baseline with the lower dose after 24 h, but an inability to return to baseline after only 6 h with the low dose; and an inability to return to baseline even after 24 h with the high dose.

In day 14 serum samples, there were also two main clusters: cluster 1 was comprised of control at 6 and 24 h (D6H, D24H) and 50 and 250 mg doses at 24 h (E24H, F24H); cluster 2 was comprised of 50 and 250 mg doses at 6 h (E6H, F6H) (FIG. 4(b)). This would suggest that when the drug was administered chronically, the rat may have a better ability to adapt and return to baseline after 24 h since the high dose rats grouped with controls.

In day 1 liver samples, there were two main clusters: cluster 1 was comprised of control (part of A6H and A24H) and 50 and 250 mg doses at 24 h (B24H, C24H); cluster 2 was comprised of part of 6 h control (A6H) and 50 and 250 mg doses at 6 H (B6H and C6H) (FIG. 4(c)). It is unclear why there was so much variation in the control group at 6 h. After 24 h, but not 6 h, the rat has restored homeostasis.

In day 14 liver, the group receiving 250 mg clofibrate at 6 h (F6H) was separated from all other groups. Again, this shows that the rats show some adaptive ability to return to baseline/homeostasis and/or ability to clear the drug more efficiently) when administered the drug chronically (14 d) as compared to a single day (1 d).

### Comparisons Between PCA And Clustering Results

The overall groupings were consistent between PCA and clustering analysis. Following single and multiple exposure to clofibrate, the control and low dose rats grouped together with subgroupings based on dose and time. High dose rats grouped separately. Rats exposed to clofibrate appeared to recover more after 24 h than after 6 h, and recovery was likely more pronounced following chronic exposure, suggesting adaptation (more efficient break down of drug, better clearance of drug, homeostatic mechanisms).

### Effects of Dose And Time Studied With GLM Statistical Approach

Effects of dose, time, and dose-time interaction were studied with a Generalized Linear Model (GLM) statistical approach. The significance of dose, time and dose*time effects was analyzed per component for d 1 and d 14 serum and liver. The two main experimental factors were dose and time. There were three dose levels (0, 50, and 250 mg/kg) and two time levels (6, 24 h), yielding six treatments. Liver and serum data collected at 48 h in Study 2 was excluded from analyses due to the confounding effect of metabolic cages inducing a stress. The GLM model equations are described below; results are shown in Chart 3.

Yᵢⱼₖ=μ+αᵢ+βⱼ+γᵢⱼ+εᵢⱼₖ Equation 1
μ: is overall mean
α is effect of i^{th} level of dose
β is effect of j^{th} level of time
γ is effect of i^{th} level of dose combined with effect of j^{th} level of time ( interaction term).
Primary hypothesis—
Ho: α₁=α₂α₃
β₁=β₂
γ₁₁=γ₁₂=γ₂₁=γ₂₂=γ₃₁=γ₃₂

CHART 3(a)
The GLM model reveals the number of significantly perturbed
(p < 0.01) components in response to time, dose and time*dose
interaction in d 1 and d 14 liver and serum samples.

| | | | | | | | Day 1 and Day 14 | | |
|---|---|---|---|---|---|---|---|---|---|
| | Day 1 | | | Day 14 | | | common components | | |
| Effect | Liver | Serum | Overlap | Liver | Serum | Overlap | Liver | Serum | Overlap |
| Time | 1771 | 892 | 7 | 1088 | 872 | n/a | 514 | 453 | 6 |
| Dose | 908 | 830 | 5 | 1263 | 713 | 4 | 355 | 377 | 3 |
| Dose*Time | 694 | 637 | 3 | 489 | 638 | n/a | 118 | 325 | 3 |

CHART 3(b)
Chart 3(b) is a derivative of Chart 3(a) focusing on the number
of components uniquely changed as a function of time, dose,
and dose * time interaction. Ratios are calculated as follows
(see bolded values in Chart 3 (a)): 1257/1771 represents the number of
components uniquely changed in day 1 but not day 14 (1771-514), divided
by the number of peaks changed in day 1 (1771). Thus, 514 peaks
(1771-1257) were changed as a function of time in both day 1 and day 14.

| | | | | |
|---|---|---|---|---|
| Effect | Liver | Serum | Day 1 | Day 14 |

| | |
|---|---|
| Day 1, not in Day 14 | Liver, not in Serum |

| | | | | |
|---|---|---|---|---|
| Time | 1257/1771 | 439/892 | 1764/1771 | 1088/1088 |
| Dose | 553/908 | 453/830 | 903/908 | 1259/1263 |
| Dose * Time | 576/694 | 312/694 | 691 | 489/489 |

| | |
|---|---|
| Day 14 not, in Day 1 | Serum not in liver |

| | | | | |
|---|---|---|---|---|
| Time | 574/1088 | 419/872 | 885/892 | 872/872 |
| Dose | 908/1263 | 336/713 | 825/830 | 709/713 |
| Dose * Time | 371/489 | 313/638 | 634/637 | 638/638 |

A large number of components were significantly changed by time, dose and dose*time interaction on d 1 and d 14 in liver and serum. There were more significantly changed components in liver than serum in some instances, but recall that there were 1.3-1.4 fold more total components identified in liver vs. serum (Chart 2). After accounting for this, in liver, there were still more components changed in d 1 in response to time, and d 14 in response to dose, compared to serum. There were a large number of components showing a significant dose*time interaction term. The interaction term indicates that dose did not have the same effect within each time point; and conversely, time did not have the same effect within each dose.

There was considerable overlap in the total number of components observed between liver and serum (whether changed by treatment or not), but Chart 3 only shows the components that were significantly changed.

### Example 8

### Identifying Early Metabolic Response To Clofibrate Exposure

Based on natural groupings of subjects, we identified metabolomic components perturbed at 6 h and returned to baseline at 24 h. Table 1 (set forth in the Detailed Description, above) shows the components at each dose and time point with significant increases and decreases relative to vehicle treated subjects. Overall, three components in serum and 13 components in liver showed a pattern of early perturbation followed by return to baseline.

### Example 9

### Putative Identification of Components, And Discriminant/Regression Analysis For Classifying Liver Hypertrophy

Liver hypertrophy is a well-known clinical end-point of chronic clofibrate exposure, however, the biomarkers of this pathology are unknown. Using the measurements of the liver weight and the body weight of the animals, we calculated the liver-to-body weight ratio (LBR). First, we conducted F-tests to determine whether the the LBR was significantly changed by dose, time, or the interaction of dose-time during Day 1 and Day 14. We found the LBR to be significantly increased in the Day 14 animals with time and with dose but not the interaction of dose-time. Second, we discovered the components that could classify the LBR in the liver and in the serum. This was accomplished out using a step wise regression method with the LBR as the response variable and all of the components as independent variables. After an iterative selection procedure, 29 of the best components from the serum and the liver were used to build the reduced model given below:

Yᵢ=β₀+β₁x₁ᵢ+β₂x₂ᵢ+β₃x₃ᵢ++β₂₉x₂₉ᵢ Equation (2)

Where Y is the LBR, β are the regression coefficients and x are the components. The 29 components for which we were able to find putative identities pathway analysis was carried out as described in Section E below.

### Example 10

### Pathway Analysis

A pathway discovery algorithm was used to elucidate possible metabolic networks spanned by the metabolites identified. Results of this pathway analysis are shown in FIG. 5. The complete list of metabolites in the figure is as follows.

1 (S)-Nororientaline

3 (S)-Norreticuline

4 (R)-Norreticuline

6 Nororientaline

7 Lysosomal-enzyme N-acetyl-D-glucosaminyl-phospho-D-mannose

8 UDP-N-acetyl-D-glucosamine

9 UMP

10 ATP

11 N6-(1,2-Dicarboxyethyl)-AMP

12 AMP

13 IMP

14 ITP

15 Arbutin 6-phosphate

16 beta-D-Glucose 6-phosphate

17 Protein N(pai)-phosphohistidine

18 N-Acetyl-D-glucosamine 6-phosphate

19 N-Acetyl-D-glucosamine 1-phosphate

20 4-(4-Deoxy-alpha-D-gluc-4-enuronosyl)-D-galacturonate

21 D-Galacturonate

22 1-Phospho-alpha-D-galacturonate

23 UDP-D-galacturonate

24 UTP

25 4-(4-Deoxy-beta-D-gluc-4-enuronosyl)-D-galacturonate

26 5-Dehydro-4-deoxy-D-glucuronate

27 CMP

28 N-((R)-Pantothenoyl)-L-cysteine

29 (R)-4′-Phosphopantothenoyl-L-cysteine

30 dTMP

31 N-Succinyl-2-L-amino-6-oxoheptanedioate

32 Succinyl-CoA

33 CoA

34 2-Oxoglutarate

35 L-Glutamate

36 N-Succinyl-LL-2,6-diaminoheptanedioate

37 Succinate

38 Fumarate

39 3-Phosphonopyruvate

40 Phosphoenolpyruvate

41 Neopterin

42 2-Amino-4-hydroxy-6-(D-erythro-1,2,3-trihydroxypropyl)-7,8-

43 2-Amino-4-hydroxy-6-hydroxymethyl -7,8-dihydropteridine

44 2-Amino-7,8-dihydro-4-hydroxy-6-(diphosphooxymethyl)pteridine

45 2-Amino-4-hydroxy-6-(erythro-1,2,3-trihydroxypropyl)

46 GTP

47 Orthophosphate

48 Pyridoxal phosphate

49 Diacylglyceryl-2-aminoethylphosphonate

50 CMP-2-aminoethylphosphonate

51 CTP

52 Diacylglycerol

53 1-Phosphatidyl-D-myo-inositol

54 N-Acetyl-D-glucosaminylphosphatidylinositol

55 Acyl-CoA

56 3-Oxoacyl-CoA

57 Phosphatidylethanolamine

58 Ethanolamine

59 Glycolaldehyde

60 5-(3′-Carboxy-3′-oxopropenyl)-4,6-dihydroxypicolinate

61 7,8-Dihydroxykynurenate

62 7,8-Dihydro-7,8-dihydroxykynurenate

63 4-Hydroxy-2-quinolinecarboxylic acid

64 4-(2-Aminophenyl)-2,4-dioxobutanoate

65 L-Kynurenine

66 Glyoxylate

67 Oxaloacetate

68 L-Aspartate

69 Glycolate

70 L-Alanine

71 (2-Aminoethyl)phosphonate

Components were associated with putative identities by comparing their M/z and chromatographic retention time against a database of known metabolites. This information was used to algorithmically generate a network of biochemical interactions to explain the observations. FIG. 5 shows a metabolic network of the 71 compounds out of which 7 are

associated with LC-MS components. Metabolite 58, ethanolamine, has been reported in the context of hepatomegaly, Thorne et al. (1994) Biochim Biophys Acta 1214:161-170. One of the putative identities for component n_{—}252.05_{—}80 is neopterin, which is a known marker of inflammation, Hoffmann et al. (2003) Inflamm. Res. 52:313-321, and of oxidative stress, Oettl et al. (2002) Curr Drug Metab 3:203-209, but has not been reported before in the context of clofibrate exposure, it is a known biomarker. Note the neopterin derivatives, 2-Amino-4-hydroxy-6-hydroxymethyl-7,8-dihydropteridine (node 43), 2-Amino-7,8-dihydro-4-hydroxy-6-(diphosphooxymethyl)pteridine (node 44) and 2-Amino-4-hydroxy-6-(erythro-1,2,3-trihydroxypropyl) (node 45). Note also that ethanolamine is involved in lipid metabolism, is regulated by peroxisome proliferators, and is associated with liver hypertrophy. Thorne et al. (1994) Biochim Biophys Acta 1214:161-170. L-Kynurenine has been implicated in severe renal failure. Pawlak et al. (2003) J. Pysiol Pharmacol 54:175-189.

### Example 10

### GC-MS Metabolomics

For GC-MS, liver was extracted with CHCl3:MeOH mixtures. Organic residue was derivitized with BSTFA and dried aqueous residues were derivitized with methoxyamine HCl/BSTFA. Samples were injected with 10:1 split into an Agilent 6890 gas chromatograph. A Leco Pegasus III TOFMS was used. Ions were generated at 70 eV with 3.2 mA ionization current; 25 spectra/s were recorded for 60-800 m/z. Acceleration voltage activated after 180 s solvent delay. Detector voltage: 1750 V. Data were processed with Leco ChromaTOF software. Automatic peak detection and mass spectrum deconvolution were performed using 1.33 s peak width. Peaks with S/N less than 20 were rejected. Component identification was accomplished with the NIST 98' MS library and, in some cases, verified with standards. Components with similarity greater than 600 were used for analysis. The results of the statistical analysis are shown in Table 3, above, in the Detailed Description.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method of determining whether a mammal has been exposed to hepatotoxant, the method comprising:
analyzing a biological sample obtained from a mammal using one or more biomarkers selected from one or more biomarkers listed in Tables 1(a), 1(b), 2, and 3, and combinations thereof; and
comparing the level(s) of the one or more biomarkers in the sample to levels of the one or more biomarkers from a control sample exposed to hepatotoxant; and
determining whether the mammal has been exposed to hepatotoxant.

2. The method of claim 1, wherein said mammal is a human.

3. A method of determining whether a mammal has been exposed to PPAR affecting drug, the method comprising:
analyzing a biological sample obtained from a mammal using one or more biomarkers selected from one or more biomarkers listed in Tables 1(a), 1(b), 2, and 3, and combinations thereof; and
comparing the level(s) of the one or more biomarkers in the sample to levels of the one or more biomarkers from a control sample exposed to PPAR affecting drug; and
determining whether the mammal has been exposed to PPAR affecting drug.

4. The method of claim 3, wherein said mammal is a human.

5. A method of determining whether a mammal has been exposed to clofibrate, the method comprising:
analyzing a biological sample obtained from a mammal using one or more biomarkers selected from one or more biomarkers listed in Tables 1(a), 1(b), 2, and 3, and combinations thereof; and
comparing the level(s) of the one or more biomarkers in the sample to levels of the one or more biomarkers from a control sample exposed to clofibrate; and
determining whether the mammal has been exposed to clofibrate.

6. The method of claim 5, wherein said mammal is a human.
